(19) United States
(12) Patent Application Publication
(10) Pub. No.: US 2010/0075339 A1
(43) Pub. Date: Mar. 25, 2010

(54) SURFACE-ENHANCED RAMAN SCATTERING (SERS) BASED NANOPARTICLE COMPOSITES

(75) Inventors: Kirk D. Rector, Santa Cruz, NM (US); Kristy L. Nowak-Lovato, Los Alamos, NM (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(73) Assignee: LOS ALAMOS NATIONAL SECURITY, LLC, Los Alamos, NM (US)

(21) Appl. No.: 12/534,612

(22) Filed: Aug. 3, 2009

Related U.S. Application Data
(60) Provisional application No. 61/137,987, filed on Aug. 4, 2008.

(51) Int. Cl. G01N 33/53

(52) U.S. Cl. 435/7.1

(57) ABSTRACT

Nanoparticle composites and method of use thereof for simultaneously sensing and probing a biological system, comprising providing a nanoparticle composite comprising a nanoparticle comprising a core and a shell; a first ligand bound to the nanoparticle, said first ligand capable of sensing pH; a second ligand bound to the nanoparticle, said second ligand distinct from said first ligand and capable of binding to a target; and bringing the nanoparticle composite into contact with the biological system to produce a first and a second pH-dependent signal; and analyzing the first or the second signal by means of surface-enhanced Raman spectroscopy.

### CROSS REFERENCE TO RELATED APPLICATION

This application claims the benefit of U.S. Provisional Application 61/137,987, filed Aug. 4, 2008, and incorporated herein in its entirety.

### STATEMENT OF FEDERAL RIGHTS

The United States government has rights in this invention pursuant to Contract No. DE-AC52-06NA25396 between the United States Department of Energy and Los Alamos National Security, LLC for the operation of Los Alamos National Laboratory.

### FIELD OF THE INVENTION

The present invention relates to nanoparticles suitable for use both in sensing and targeting in a cellular or whole organism, for example, sensing a chemical environment such as pH in an endosomal pathway in a given cell type, and methods of use thereof.

### BACKGROUND OF THE INVENTION

Examining the inner-workings of a live biological cell is a complex task. There exist many tools that can be used to probe various areas or states within the cell, which generally fall within one of two categories. The first category includes probes which can target, either directly or indirectly, a specific location, structure, or biochemical class of molecules, such as specific proteins, which then may be used to create contrast in cellular microscopy. Directly labeling the cell can be accomplished in fluorescent microscopy where an antibody is attached to a fluorophore or a quantum dot, or in electron microscopy where the antibody is attached to a metal nanoparticle. Direct labeling can also be accomplished using the genetic sequence during in situ hybridization. Indirect labeling, although more cumbersome, can be accomplished with a primary antibody being detected by a secondary antibody conjugated to a fluorophore, quantum dot, and the like. Labeling is a highly useful technique to target and visualize the location of specific structures within the cell. The second category includes sensors capable of detecting dynamic chemical changes occurring within these live cells. Sensors are available to measure many kinds of ions from metal ions such as Zn²⁺, Mg²⁺, Ca²⁺, or ions such as Na⁺, Cl⁺, K⁺, for monitoring oxygen levels and for pH measurement.

The numerous fluorescence probes that are commercially available have dramatically increased the researcher's ability to probe cell structure, organelles, movement, chemical makeup, and response to stimuli. Generally the signal transduction of these probes involves a fluorescence spectral change upon binding of the target. The analyte concentration affects the magnitude of this spectral change. However, these probes suffer from interactions with other molecules within the cell, leaching out of the generalized measurement area, and photobleaching, which limit some applications, especially changing concentrations over time. The wide spectral response of these fluorescence probes also creates a practical limit in the number of independent labels that can be simultaneously measured without overlap of about three to four in the visible range. The use of nanocrystal quantum dots, with their much narrower spectral response, has increased the number of simultaneous measurements that can be performed to approximately ten, utilizing both the visible and near infrared spectral ranges.

Fluorescence sensors responsive to a variety of analytes have been used in many cell types. Generally, features in the spectrum change in response to the changing analyte concentration, chemical identity, or oxidative state. In addition, fluorescence resonance energy transfer (FRET) measurements can be included in this class of experiments, although the spectral change seen is from two different chromophores. These types of experiments have driven the development of multispectral fluorescence microscopy systems, with tens of spectral channels able to record the spectral changes of the fluorescence sensors as they respond to the cellular environment. There also exist a variety of organic dyes and fluorescent proteins sensitive to changes in cellular acidity. Perhaps the most commonly used reagents are various fluorescein derivatives, including 2′,7′-bis(2-carboxyethyl)-5-carboxyfluorescein (BCECF), and the LysoSensor™ family. All of these sensors are themselves weak acids or bases. At the concentrations necessary for imaging, they can perturb the pH of the cell, which is a severe limitation. Therefore, these pH-sensitive dyes are useful for labeling and tracking acidic organelles rather than for precisely measuring intracellular pH over time.

It is also possible to perform similar experiments using Raman scattering as the contrast mechanism in cellular imaging. Because the spectral response of spontaneous Raman scattering processes is typically narrower than the typical spectral response of fluorescence or quantum dot species, the ability to make multiple measurements simultaneously is greatly enhanced. Raman spectroscopy provides chemically specific information based upon unique vibrational signatures of the species present. Therefore, recording of the spectra of a cell can give detailed information about the chemical species present. Unfortunately, the Raman effect is weak, with molecular cross-sections on the order of 10²⁹ to 10³² cm², which is significantly lower than those of good fluorescence labels, which are on the order of 10¹⁶ cm² range. The small cross-sections for Raman scattering necessitate long integration times, which hinder measurements of dynamic systems or require intense excitation power, which in turn can create sample damage. In addition, electronic transitions found in native compounds often overlap with these excitation wavelengths, leading to unwanted background fluorescence (i.e., autofluorescence) that can obscure relatively weak Raman signals.

There exists a need, therefore, for sensors and probes that have the sensitivity of fluorescent molecules, which have the narrow spectral response of Raman scattering so as to enable multiple analyses, and which do not perturb the cellular response systems.

### SUMMARY OF THE INVENTION

The present invention describes a method of simultaneously sensing and probing a biological system. The method utilizes nanoparticle composites that have both a targeting component as well as a sensing component. The method and nanoparticle composites are described in part in Nowak-Lovato et al., “Targeted Surface-Enhanced Raman Scattering Nanosensors for Whole-Cell pH Imagery,” Applied Spectroscopy, v. 63, n. 4, pp. 387-395 (2009), incorporated herein by reference in its entirety. SERS is a sensitive tool for exploring metal-adsorbate interactions and reactivity of adsorbed species and provides an increase in the Raman scattering signal by several orders of magnitude. This enhancement makes it possible to access the chemical specificity inherent in Raman spectroscopy with low detection limits and/or fast acquisition speeds. The nanoparticle composites have an ability to scatter visible light efficiently, thus allowing for multiple analyses. The nanoparticle composites are capable of targeting, e.g., through the FcεRI receptor-mediated endocytic pathway due to the adsorption of 2,4-ε-dinitrophenol-L-lysine (DNP) ligand on the nanoparticle surface. The nanoparticle composites are sensitive to pH changes in the endocytic vesicle within the pH range of 4.0-8.0 by, e.g., 4-mercaptopyridine (4-MPy) adsorbed to the particle surface. The targeting and sensing moieties of the present invention do not significantly interfere with each other's function. The targeted nanoparticle composite further is capable of making accurate cellular pH measurements in RBL-2H3 cells, up to ninety minutes after targeted nanoparticle addition. Thus, the method of the present invention provides a means for providing information, such as pH, both spatially and temporally, and in response to chemical and physical stimuli.

The present invention is distinguished from previous attempts at sensing and probing biological cells in that the method is suitable for use with whole, live cells, and can provide simultaneous, spatial information about both pH and nanosensor location at sequential time points.

The following describe some non-limiting embodiments of the present invention.

According to one embodiment of the present invention, a method of simultaneously sensing and probing a biological system is provided, comprising providing a nanoparticle composite comprising:
i. a nanoparticle comprising a core and a shell;
ii. a first ligand bound to the nanoparticle, said first ligand capable of sensing pH;
iii. a second ligand bound to the nanoparticle, said second ligand distinct from said first ligand and capable of binding to a target;
   bringing the nanoparticle composite into contact with the biological system to produce a first and a second pH-dependent signal; and analyzing the first and the second signal by means of surface-enhanced Raman spectroscopy.

According to another embodiment of the present invention, a nanoparticle composite is provided, comprising a nanoparticle comprising a core and a shell; a first ligand bound to the nanoparticle, said first ligand capable of sensing pH; and a second ligand bound to the nanoparticle, said second ligand distinct from said first ligand and capable of binding to a biological target.

According to yet another embodiment of the present invention, a nanoparticle composite is provided, comprising a nanoparticle comprising a noble metal; a first ligand bound to the nanoparticle, wherein the first ligand is 4-mercaptopyridine; a second ligand bound to the nanoparticle, wherein said second ligand is a 1,2-dinitrophenol-lysine conjugate; wherein the nanoparticle composite is suitable for use in analysis by surface-enhanced Raman spectroscopy.

### BRIEF DESCRIPTION OF THE DRAWINGS

FIG. 1 depicts a schematic representation of the nanoparticle composite of the present invention comprising a nanoparticle having a gold core and a silver outer shell with DNP and 4-MPy ligands (not drawn to scale).

FIG. 2 shows normalized and offset Raman spectra during in vitro sensor development: (A) Raman spectrum of colloids comprising nanoparticle composites, wherein the nanoparticles have a gold core and a silver outer shell; (B) Raman spectrum of 4-MPy adherent to colloids comprising nanoparticle composites having a gold core and a silver outer shell; (C) Raman spectrum of DNP adherent to colloids comprising nanoparticle composites having a gold core and a silver outer shell; and (D) Raman spectrum of nanoparticle composites with both DNP and 4-MPy ligands. The y-axis is arbitrary intensity units.

FIG. 3 shows surface-enhanced Raman scattering (SERS) spectra of a 0.5 μm×0.5 μm sized pixel in vivo, demonstrating endosomal pH. The spectrum was taken with 1.0 s integration. pH values are: (A) 4.5, (B) 5.7, (C) 6.5, and (D) 7.7. The y-axis is in arbitrary intensity units.

FIG. 4 shows in vivo calibration curve of the nanoparticle composites of the present invention inside RBL-2H3 cells. The y-axis represents the ratio of the integration of the peak centered around 1580 cm⁻¹ versus the additive integration of both the peaks centered near 1580 cm⁻¹ and 1612 cm⁻¹. Horizontal error bars are dependent on accuracy of the pH meter. Vertical error bars are dependent on approximately 100 pixels per image, repeated five times.

FIG. 5 shows whole-cell image data ranging from 3.5 to 90 minutes. Total nanoparticle composites intensity per image is depicted with the gray line, corresponding to the primary y-axis. Total pixel count per image is shown with the black line, corresponding to the secondary y-axis. Average intensity per pixel is displayed with the dashed line and also corresponds to the secondary y-axis.

FIG. 6 shows the fraction (y-axis) of pixels representing each pH grouping over time (x-axis). Images were acquired from 3.5 to 90 minutes, taken every 3.5 minutes.

### DETAILED DESCRIPTION OF THE INVENTION

The present invention described a method of simultaneously sensing and probing a biological system. Herein, “biological system” is understood to mean an animal, plant, bacteria, algae, or other whole organisms, as well as individual cells, groups of cells, or cellular components.

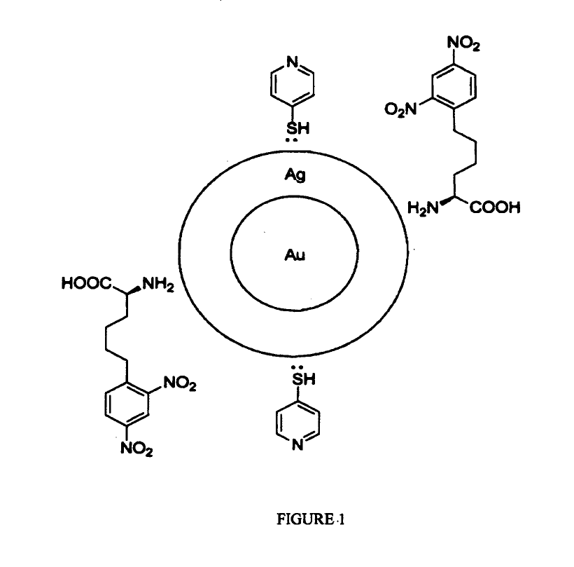

The present invention describes nanoparticle composites which have both the ability to target to a specific biochemical pathway and to sense the concentration of pH at this location is presented. The nanoparticle composite is compatible with surface enhanced Raman spectroscopy (SERS). Spontaneous Raman spectroscopy or imaging provides chemically-specific information based upon unique vibrational signatures of the target analyte which in turn is based upon its chemical structure. Although Raman is a technique with high specificity, signal strengths are low. Signal enhancement can be achieved by conjugating the molecule of interest to a roughened metal surface. The nanoparticle composites comprise three components: 1) a base metal nanoparticle; 2) a ligand bound to the nanoparticle capable of sensing the analyte under study and 3) a ligand bound to the nanoparticle capable of targeting the nanocomposite to desired cellular location. One arrangement of the components of a nanoparticle composite of the present invention is depicted in FIG. 1 (analyte not shown). The nanoparticle composites sense changing pH values, while being targeted through the endocytic pathway.

The nanoparticle composites of the present invention must be suitable for use in surface enhanced Raman spectroscopy analysis, meaning that they must provide sufficient sensitivity, and may not interfere with the analysis to any significant extent. The nanoparticle composites may comprise nanoparticles having a core substantially surrounded by a coating. The core and/or coating may comprise metals such as gold, silver, platinum, copper, and combinations thereof. Alternatively the core may comprise dielectric materials or air (i.e., be hollow). In one embodiment, the core comprises gold and the coating comprises silver. The coating may be a substantially continuous layer or may be comprised of smaller particles which together substantially coat the core. Alternatively, the nanoparticles may be solid, shells, pyramids, or composites, wherein a composite is understood to mean a nanoparticle in which the entire particle, the core and or the shell comprise a non-metal material such as a polymer. Non-limiting examples of suitable composite nanoparticles suitable for use as SERS substrates are described in U.S. Patent Application 2008/0171656, Wang et al., filed Jan. 11, 2007. The diameter of the nanoparticle may be from about 20 nm to about 500 nm.

The pH sensing ligand can be any ligand capable of sensing changes in pH, which can be stably bound to the nanoparticle composite and which does not interfere with the ability of the targeting ligand to bind to its target. In principle, a variety of weak acids or bases may be used to sense pH. However, the ligands should have a mechanism to bind tightly to the nanoparticle surface, e.g., through sulfur-metal or nitrogen-metal bonds. In one embodiment, multiple sensing ligands may be used to detect multiple analytes simultaneously, or to detect a single analyte across wider ranges in concentration than would be detectable by a single sensing ligand. One example of a suitable pH sensing ligand is 4-mercaptopyridine (4-MPy), which demonstrates ratiometric diagnostic frequency modes near 1600 cm⁻¹, depending on the concentration of [H⁺] bound or unbound to pyridine. In the present invention, the sensed pH is from about 4.0 to about 8.0. However, it is understood that other ligands may be used which would extend the range of sensed pH to from about 1.0 to about 12.0.

The targeting ligand, or targeting agent, may be any of a wide variety of ligands useful for targeting a particular organism, cell structure, or molecule, and which can be stably bound to the nanoparticle composite and which does not interfere with the ability of the pH sensing ligand to sense an analyte. For example, carbohydrates or antibodies may be used for targeting, and in one embodiment, the targeting, or second, ligand is an antibody-based targeting agent or a carbohydrate based targeting agent. One example of a suitable targeting ligand for FcεRI mediated endocytosis is 1,2-dinitrophenol (DNP) conjugated to lysine, an antigen for IgE endocytosis. Accordingly, one target of the present invention are IgE receptors. Alternatively, the target may comprise positive and negatively charged ions, such as Ca⁺², Fe⁺², Cl⁻, and small molecules, such as NO and CO.

### Method

The present invention provides a method of simultaneously sensing and probing a biological system. Herein, “simultaneously” is understood to mean substantially at the same time, and within the timeframe of a single experiment. The method comprises the step of providing a nanoparticle composite as described herein. The nanoparticle composite is then brought into contact with a biological system, where the nanoparticle may target a specific cellular location or biochemical pathway. At this location or pathway, the sensing ligand detects the analytes' localized concentration. When an analyte is sensed, a first signal pH-dependent signal is produced. When a chemical moiety is targeted, a second pH dependent signal is produced. One or both of the signals may be detected and analyzed by appropriate analytical instrumentation. In one embodiment, the signal(s) are detected by surface-enhanced Raman spectroscopy. The sensing and probing may occur intracellularly and/or on the cell surface. In one embodiment, the target and the analyte are measured intracellularly, with the cell intact.

In the present method, the pH-dependent signal may change in response to external stimuli. The external stimuli may be physical conditions, such as temperature, or may be chemical conditions. Examples of chemical conditions may include the presence of drugs such as amiloride or bafilomycin.

### Examples

Chemicals and Materials. Gold colloids with a diameter of 60 nm were purchased from Ted Pella, Inc. (Redding, Calif.) at 2.6×10¹⁰ particles per mL. The Au colloids were enhanced with an Ag coating by addition of LI silver-enhancing and initiator agents from Nanoprobes, Inc. (Yaphank, N.Y.). Silver enhancement was performed at a 1:1 ratio of both initiator and enhancer. The enhancement solution was added to the colloids for 2 minutes, centrifuged at 13,000 rpm for 5 minutes and then washed with deionized H₂O. 4-Mercaptopyridine (4-MPy) (Sigma Aldrich Chemicals, St. Louis, Mo.) and 2,4 ε-dinitrophenol-L-lysine (DNP) (MP Biomedical, Solon, Ohio) were used without further purification and aqueous solutions were prepared at 1 mM. BSA-DNP and IgE antibody was acquired via a generous gift from the Oliver-Wilson laboratory at the University of New Mexico. Nigericin, KCl, MgCl₂, CaCl₂, and glucose were purchased from Sigma Aldrich chemicals (St. Louis, Mo.) and used without further purification.

Cell Culture. For cellular experiments, RBL-2H3 cells were cultured in MEM media supplemented with 10% fetal clone III (Thermo Scientific Hyclone, Logan, Utah), 90 IU/mL of penicillin (ATCC, Manassas, Va.), and 90 μg/mL streptomycin (ATCC, Manassas, Va.), with an additional 2 μM of L-glutamine (Sigma Aldrich, St. Louis, Mo.). RBL-2H3 388 cells are an adherent cell line that was grown in a 5% supplied CO₂ incubator at 37° C. to 80% confluency and then passaged. IgE antibody is added to the cells for sensitization 24 h prior to use at 1 μg/mL and kept at 37° C. and 5% CO₂. Imaging experiments with the targeted nanocomposites employ sonication of colloids for five minutes, filtration through a 0.2 μm filter, and addition to the cells at 10-100× particles per cell number. After 5 minutes, the cells are then washed 2× with phosphate buffered solution to eliminate most of the residual nanoparticles left outside of the cell, and then freshly warmed Hanks buffer is applied. The buffer comprises Hanks, HEPES, BSA, glucose, MgSO₄, and NaHCO₃. The cover slips with adherent cells are then added to the environmental chamber vessel (Carl Zeiss, Inc., Thornwood, N.Y.) equilibrated at 25° C. and 5% CO₂ for imaging.

Degranulation Assay. To demonstrate biological activity of the IgE receptor mediated endocytosis nanocomposite, a degranulation assay was performed. In this assay, β-hexosaminidase was used to cleave hydrolytically the substrate p-nitrophenyl-N-acetyl-β-D-glucosaminide (N9376, Sigma) and cause a color change that can be detected. The assay was performed in 24-well culture plates. Positive control for the assay is done by lysing whole cells with a 1% Triton solution.

Microscope Layout and Imaging. The microscopic system can be used to collect bright-field, fluorescence emission, Raman scattering, and ultraviolet-visible (UV-Vis) absorbance spectral images, depending upon slight modifications to the setup. The microscope imaging system is based upon a Carl Zeiss Axiovert 135TV inverted microscope with an Epiplan 103(N.A., 0.20), a LD Plan-achroplan 40x, (N.A. 0.60) and a C-apochromat 63x (N.A. 1.2) water immersion objective. It was this last objective that was primarily used for the SERS experiments. A xenon arc lamp (XBO 75, Carl Zeiss, Thornwood, N.Y.) was used to illuminate the sample for bright-field visualization in transmission mode, by video camera, using transfer optics in the trinocular head (Optem 70XL, Labtek, Campbell, Calif.). The total zoom factor of the transfer optics was 0.53 in addition to the objective used. An Infinity 2-2 monochrome (Lumenera Corp., Ottawa, ON) camera was used to acquire the visible image using an acquisition rate of approximately 200 ms/frame and no detector gain. The visible image resolution was 1616×1216 pixels. The camera was connected to the personal computer through a USB 2.0 connection and the image was captured using the Infinity Capture software and saved as a .tiff file. Raman experiments were performed using 514.5 nm light from a Spectra Physics 177-G01 air-cooled argon ion laser. The excitation light was separated from other Ar⁺ laser lines using a 300 lines/mm dispersive grating and an approximately 1 mm spatial filter several feet away. The laser light is expanded and collimated using a 1:2 Keplerian telescope. It is coupled into the back of the microscope objective with an antireflection-coated plano-convex BK 7 150 mm focal length cylindrical lens (CKX150AR.14, Newport Corp. Irvine, Calif.), a Raman edge dichroic (z514rdc #105366, Chroma Technology, Rockingham, Vt.), and a 50.8 mm diameter, 150 mm focal length, biconvex spherical antireflection coated doublet tube lens (PAC086AR.14, Newport Corporation, Irvine, Calif.). The laser light is focused to a line approximately 0.5 μm wide and 50 μm tall and a total power at the sample of 10 mW for the experiments detailed in this paper. The Raman signal is captured with the same objective and tube lens and is passed through the edge dichroic. The signal is transferred using two additional 150 mm spherical lenses and re-imaged onto the 100 μm wide slit of a 0.25 m f/2.2 imaging spectrograph (Holospec 2.2, Kaiser Optical Systems, Inc, Ann Arbor, Mich.). The signal is dispersed by a volume holographic grating (HSG-514.4-LF,

Kaiser) and imaged with a LN₂ cooled CCD array detector (LN/CCD-1024E, Princeton Instruments, Trenton, N.J.). Each image of the CCD camera records spectral and “Y” spatial information of the sample. The spectral calibration of the CCD was performed using a Ne standard lamp (Neon 6032, Newport, Irvine, Calif.) and confirmed with an Hg standard lamp (Newport). The spectral resolution of the spectrograph is approximately 2 cm⁻¹. The spatial axis was calibrated using a resolution test target, USAF-1951 using lines in the 6^{th} group. The spatial axis was corrected for spherical aberrations using the Hg standard lamp and Labview code (National Instruments, Austin, Tex.). In addition, a flat-field correction was performed to remove the roughly Gaussian shaped power dependence along the laser line focus using a NIST Relative Intensity Correction Standard (NIST 2243). Individual spectra were acquired by integration along the vertical axis and background spectra were acquired under the same conditions using a microscope coverslip only. The data were acquired using Labview code and stored in ASCII format on a personal computer. In order to acquire spectral image cubes, the frequency and “Y” spatial axis was acquired and saved, and the sample was moved across the excitation source by stepping the computer-controlled microscope stage (MS-2000, Applied Scientific Instrumentation, Eugene, Oreg.), to sequentially build up the “X” spatial axis of the cube. The synchronization of the CCD camera and the stage were performed using the same Labview code. All cellular experiments were performed with an independently controlled environmental chamber (Carl Zeiss, Inc., Thornwood, N.Y.) mounted to the stage.

Surface-Enhanced Raman Scattering pH Calibration. In vivo calibration experiments employ the addition of 10 μm nigericin, 140 mM KCl, 1 mM MgCl₂, 2 mM CaCl₂, 5 mM glucose, and 20 mM phosphate-citrate buffers of varying pH (4-8). Once the calibration solution was added, cells were returned to the incubator for ten minutes. After pH equilibration, targeted nanocomposites were added to the cells and whole images were taken of all particles found to gain entry inside the cells after ten minutes of addition. Cellular images representing approximately 100 0.5 μm×0.5 μm sized pixels with nanocomposites were used. These images were processed to obtain individual spectra as indicated in the data analysis section. Once individual data points were collected for each pH value, the data were fit to a sigmoidal curve.

Data Analysis Procedures. The data analysis procedures were performed using Labview code. All pixels of the hyper-spectral image cube were subject to the same correction procedures including wavelength calibration, flatfield correction, background subtraction, CCD bias voltage subtraction, and spherical aberration removal. The images were thresholded by eliminating any spectral pixel information with a signal-to-noise ratio of less than 2.5. The signal-to-noise ratio was calculated by taking the highest intensity count from the integrated peak at 1572 cm⁻¹ to 1599 cm⁻¹ subtracted from the baseline value at that point, divided by the highest intensity count from the noise at 1800 cm⁻¹ to 1827 cm⁻¹ subtracted from the baseline value at that point. Any pixel with a signal-to-noise ratio below 2.5 was considered to have no useful information and was removed from the image. The two spectral peaks of interest were then integrated from 1572 cm⁻¹ to 1599 cm⁻¹ for the non-protonated peak and 1599 cm⁻¹ to 1626 cm⁻¹ for the protonated peak. Because the Raman spectra often occur on a weak fluorescence background, the integrated limits were evaluated against a linearly sloping background defined as the spectral intensity at 1000 cm⁻¹ and 2000 cm⁻¹. Because the peaks of interest are so close spectrally, variably sloping baselines that may arise from the cellular autofluorescence can be effectively removed without changing the intrinsic ratio of the peaks. Unlike other pH sensors used, the 4-MPy sensor has the additional benefit of having all the ligand molecules in either one state or the other. Thus, the peaks are plotted not as a ratio to a standard peak, but rather as a percentage of molecules in the un-protonated state (Table I).

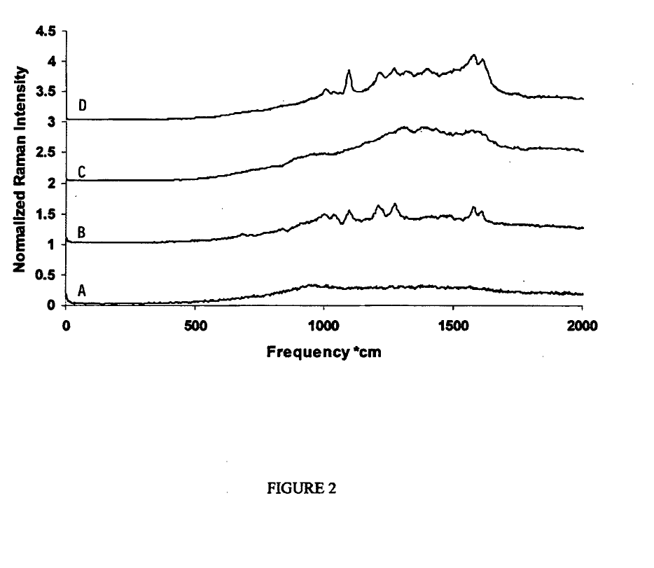

FIG. 2 shows a SERS spectrum of unmodified nanoparticle composites having a gold core and a silver outer shell (A), which has no significant Raman signatures. (B) shows the SERS spectrum of nanoparticle composites having a gold core and a silver outer shell with 4MPy, which shows a variety of peaks including the peaks near 1600 cm⁻¹ of primary interest and generally agrees with those reported elsewhere. (C) represents the SERS spectrum of nanoparticle composites having a gold core and a silver outer shell with DNP, which demonstrates weak Raman peaks; however, these peaks are of a decreased intensity most likely due to the fact that DNP is conjugated to lysine and is further in distance from the nanoparticle surface. The distance of DNP from the particle could be contributing to the weakened DNP Raman signals. (D) represents a SERS spectrum of nanoparticle composites having a gold core and a silver outer shell with both 4MPy and DNP that shows general agreement with (B) with a mild contribution from DNP.

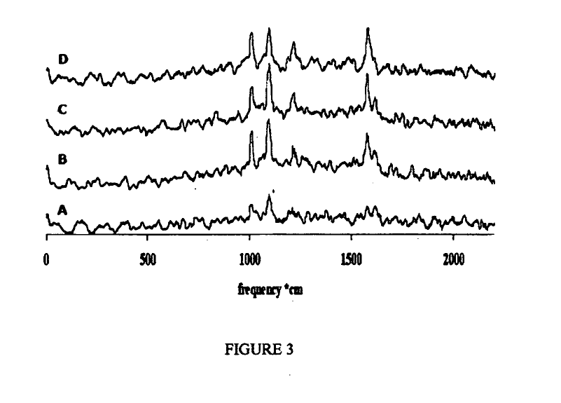

FIG. 3 illustrates representative averaged Raman spectra of 4MPy-DNP bound to individual nanoparticle composites having a gold core and a silver outer shell. The pH values of the spectra are (A) 4.5, (B) 5.7, (C) 6.5, and (D) 7.7. It should be noted that many peaks associated with 4-MPy seem to display a certain degree of pH sensitivity. In particular, vibrational modes at frequencies of 1580 cm⁻¹ and 1612 cm⁻¹ display proportional ratiometric affects dependent upon pH. These vibrational modes are based on protonation and deprotonation of the ring N atom in the 4-MPy molecule. Protonation of the molecule has been assigned to the vibrational mode at 1612 cm⁻¹ while the unprotonated form has been assigned to 1580 cm⁻¹. The peaks do not appear to shift spectrally with changing pH in this range. Calculating the ratio between the two frequency modes demonstrates a value that can be correlated to the pH of the molecule's environment. Since the two peaks to be ratioed are spectrally close and completely dependent upon the same element (nitrogen) and whether it is bound or unbound to hydrogen, this sensor can accurately report pH without being tainted by the quantity of molecules present or by other molecular bond orientations. As is demonstrated, the intensity of the 1612 cm⁻¹ peak is greatest at low pH and decreases while the pH is increased. The peak at 1580 cm⁻¹ is greatest at high pH and decreases with decreasing pH. The spectra are representative of an individual pixel, inside the cell, at 1.0 s integration with an average power at the pixel of approximately 0.1 mW. The signal-to-noise ratio of these data is decreased by the need to acquire whole-cell imagery at fast acquisition times. Raman spectra acquired at low pH values such as 4.5 and below tend to be noisier than those at alkaline pH values. The decrease in signal could be affected by decreased 4-MPy adsorption to the nanoparticle surface, interactions with other molecules at the pyridine group, or molecular interactions caused by adjacent DNP. Thus, for these endocytic sensors, pH measurements below 4.0 are not reliable.

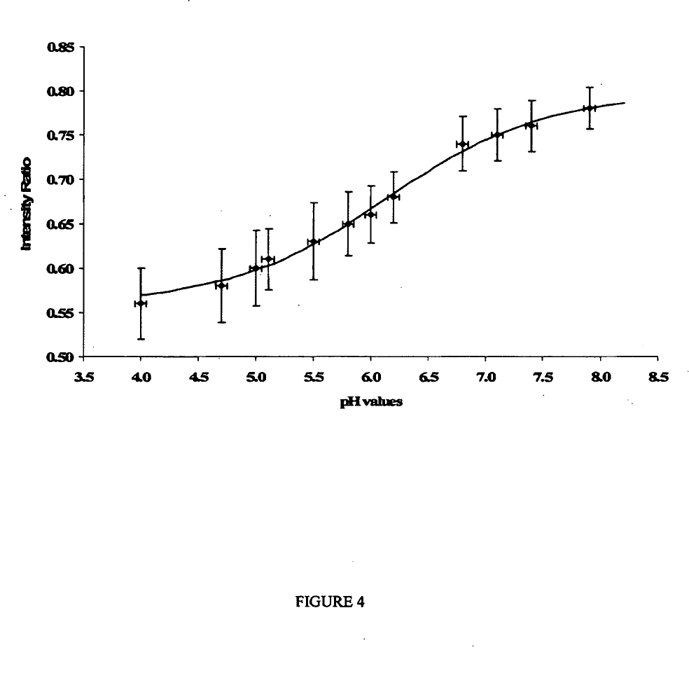

Using many measurements of this type, the in vivo calibration data are shown in FIG. 4. The error bars for pH demonstrate an estimated error of about 0.1 pH units using a calibrated IQ Scientific pH meter to make accurate pH measurements when the pH was measured four independent times. The error bars for the frequency ratios represent the standard deviation of the repeated measurements and generally are more precise at higher pH values. Vesicles formed along the endocytic pathway have been shown to display pH concentrations from 4.5 to 7.5. This range agrees quite well with our calibration curve, showing a relevant pH range from 4.0-8.0 pH units. These data were fit to a sigmoidal curve with the values shown in Eq. 1. The following equation was used to convert SERS spectra to pH maps in the subsequent experiments:

y=0.80−0.24{1+exp [(x−6.1)/0.69]}

To test the effect of the presence of 4-MPy on the ability of a DNP particle to be targeted to the endosomal pathway of cell lines, a fluorescence-based degranulation assay was performed.

Cellular degranulation upon stimulation of the IgE FceRI mediated pathway will result in the cell's secretion of constituents including the granular enzyme β-hexosaminidase. Release of β-hexosaminidase is used in this assay to hydrolytically cleave an externally added substrate molecule, p-nitrophenyl-N-acetyl-(3-D-glucosamine), and cause a detectable color change. An assay was used to compare nanoparticle composites having a gold core and a silver outer shell with DNP with previously measured bovine serum albumin (BSA)-DNP particles. Assay data are given in Table I, where the % Release column relates the percentage of fluorescence signal measured in a sample compared with the control of the signal from lysed cells. Spontaneous release resulting from IgE sensitized cells, without the addition of BSA-DNP, shows a response at 5%, which is used as the negative control. A 5% release comparable to spontaneous release was demonstrated with the use of nanoparticle composites having a gold core and a silver outer shell with 4MPy, lacking the targeting DNP component. Decreasing concentrations of BSA-DNP show a corresponding drop in percentage release, which serves as a positive control and generally agrees with literature values for this cell line. Nanoparticle composites having a gold core and a silver outer shell with DNP show a 55% granule release from whole cells, which is comparable to that seen with 10 μg of BSA-based DNP particles. When DNP:4-MPy mixtures of 1:1, 3:1, and 1:3 were used to coat the nanoparticle composite surface, degranulation percentages were seen at 30%, 37%, and 27%, respectively, which is comparable to 10 ng of BSA-based DNP. This drop in efficiency can be attributed to the decrease in concentration of the DNP on the particle surface because of the presence of 4-MPy. The 1:1 ratio (DNP:4-MPy) nanoparticles was chosen for these experiments as a reasonable balance between the ability of DNP to target the nanocomposite to the endosomal pathway, with high enough SERS signal levels from 4-MPy to enable reasonably fast image acquisition.

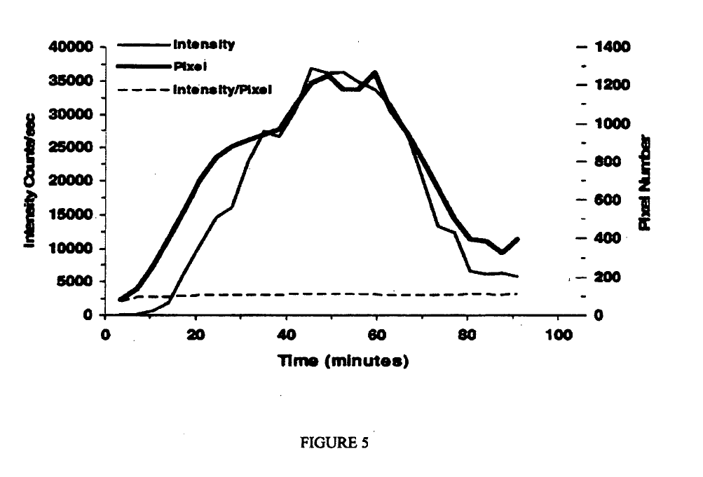

The number of pixels having significant SERS signal are plotted in FIG. 5, representative on the secondary y-axis. There is most likely not a one-to-one correspondence between pixels measured and particles internalized. The measured size of each pixel is 0.5 μm×0.5 μm, which is also roughly the size of endosomes seen in this cell type. The targeted nanocomposites are below 0.2 μm in size. Therefore, the targeted nanocomposites encompass single pixels on average. However, it is uncertain whether multiple nanocomposites are present within an individual pixel. At fifteen minutes, approximately 400 pixels have SERS signal above the threshold. Over the course of an hour, the number of pixels with SERS signal swells to its peak at approximately 1200. Over the subsequent thirty minutes, the number of pixels measured decreases to just fewer than 400. The rise in pixels over the first hour after nanocomposite delivery is explained by the slower cell processes expected to occur at 25° C. compared to the normal cellular growth temperature of 37° C. The nanocomposites delivered suggest that the washing step did not completely remove free nanocomposites. The drop in total pixels after one hour could possibly result from the degranulation process, where the nanocomposites are excreted from the cells over time. In addition, it is possible that some of the nanocomposites are being destroyed by the interrogation of the laser or being chemically modified by the harsh environment of the endosome. The possibility also exists that the nanocomposites are aggregating, causing a decrease in pixel number without a loss of nanocomposites.

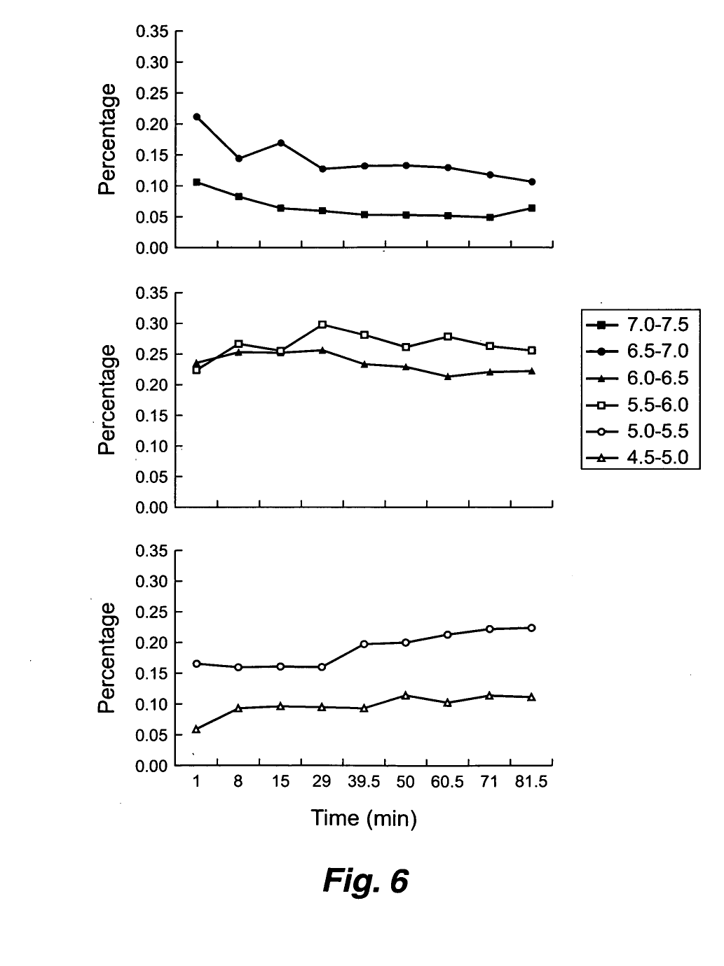

The percentages of particles within pH categories that correspond to hypothetical states in the endosome maturation pathway are recorded over time. These data are illustrated in FIG. 6. The pH of the media used is approximately 7.4. It is assumed that immediately after internalization the endosome would have the same pH as the surrounding media. In the cell under interrogation, immediately there are about 9% of the pixels in the pH=7.0-7.5 category and 17% of the pixels in the pH=6.5-7.0 category. In both categories, the percentages of particles in these categories drop by 90 minutes to 6% and 11%, respectively. This trend can be attributed to internalized endosome maturing to lower pH values and recycled receptors having fewer particles available for new internalization processes. The particles representing the category of pH=6.0-6.5, for the early endosome, begin the experiment at 17% of the population then rise to 22-23% of the population at 15 minutes. The percentage of this endosomal population remains nearly constant as particles entering and leaving this pH range are nearly balanced. After seventy minutes, the population begins to drop, as there is a smaller percentage of endosomes entering this category from the pH 7 range. The category of pH=5.5-6.0, representing possibly the sorting endosome/early endosome, is the most populous category; the population begins in the high twenties and remains constant throughout the experiment. The categories of pH=5.0-5.5 and pH=4.5-5.0, representing the late endosome/lysosomal stage, begin the experiment at 19% and 9%, respectively. Over time, both categories increase in relative abundance, ending at 23% and 12%, respectively. This increase in abundance suggests that the bulk of the particles have been processed to the late endsome/lysosomal stage.

TABLE I
β-Hexosaminidase assay table of results.

| Sample | % Release |
|---|---|
| Spontaneous | 5 |
| 10 mg BSA-DNP | 50 |
| 1 mg BSA-DNP | 47 |
| 10 ng BSA-DNP | 37 |
| 1 ng BSA-DNP | 26 |
| Au@Ag-DNP (100%) | 55 |
| Au@Ag-DNP (50%:50%) | 30 |
| Au@Ag-DNP (75%:25%) | 37 |
| Au@Ag-DNP (25%:75%) | 27 |

### Physical and Chemical Stimuli—Temperature

### Bafilomycin and Amiloride

Bafilomycin (Sigma) was dissolved in EtOH and used at a concentration of 200 nM for cell experiments. Amiloride (MP Biomedical, Inc., Solon, Ohio) was dissolved in distilled H₂O and used at a concentration of 1 mM for cell experiments. The compounds were added to the media or Hanks buffer, and then added to the cells at the specified time points within each individual experiment.

TEM imaging showed high resolution imaging of the internal structures within the cell along with the ability to see individual nanosensors. After a five minute pulse of nanoparticle composites as shown in FIG. 1, a nanosensor was seen bound to a fingerlike projection of the cell membrane. Incubation of the nanosensor for a five minute pulse followed by a sixty minute chase showed four nanosensors inside a cellular vesicle compartment and a fifth nanosensor localized in another cellular vesicle compartment. Three nanosensors were seen in a cellular vesicle compartment within the cell. The TEM images show that the nanosensors were internalized from the surface and localized to cellular vesicle compartments, believed to be trafficking through the endocytic pathway. The use of TEM demonstrates a high resolution image showing internalization of the nanosensors within cellular vesicles.

The pH sensitivity of the nanosensor, to less than 0.1 pH units, has been demonstrated above with in vivo pH calibration of cells by internal and external equilibration of pH. An in vivo pH calibration curve from pH 4.0-8.0 of the nanosensors inside RBL-2H3 cells also has been demonstrated in Nowak-Lovato et al., “Targeted Surface-Enhanced Raman Scattering Nanosensors for Whole-Cell pH Imagery,” Applied Spectroscopy, v. 63, n.4, pp. 387-395 (2009).

The nanosensors may be colored to represent the pH of their cellular environment. Each colored pixel represents nanosensors that correspond to their Raman signature. Using the previously mentioned calibration curve, the Raman signature will coincide with a specific pH value. Once the pH of the nanosensors is established, it is colored according to a rainbow-colored calibration bar.

To enable communication of such a large data set, pH groupings may be used to categorize the nanosensors and enable visualization of the trends that occur over time. The pH groupings were identified as a means of characterizing endocytic vesicles under normal cellular condition. Trafficking through the endocytic pathway involves specialized endosomal vesicles with specific pH values. A vesicle that has just invaginated from the cellular membrane, known to be a pre-endosomal vesicle, will be within the 7.0-7.5 pH range. A vesicle that is in the recycling endosome would be expected to be within the 6.5-7.0 pH grouping. An early sorting endosome would have lower pH and can be expected to be within the 6.0-6.5 pH grouping. The late endosome has been thought to have a pH range from 5.0-6.0 therefore these vesicles could be in the 5.0-5.5 or 5.5-6.0 pH grouping.

At 37° C. it has been observed that once FcεRI receptor cross-linking has occurred, 50% of maximal endocytosis of the receptors will occur within 5-13 minutes and most will be trafficked to lysosomes within 30 minutes. The nanosensor groupings devised from images (not included) show that at twelve minutes, 90% of the nanosensors are in pH groupings indicative of late endosomal/lysosomal vesicles. The pH groupings at twenty-one to thirty minutes demonstrated a reduction in the previous pH groupings to 75-80% of nanosensors localized to late endosomal/lysosomal vesicles. An increase in nanosensors grouped into pH groupings of 6.0-7.0, indicative of early sorting endosomes and recycling endosomes, was also seen between twenty one to thirty minutes.

Cells incubated at 37° C. were compared to cells undergoing endocytosis at 25° C. At 25° C. endocytosis progresses on a much slower time-scale. The nanosensors categorized into the recycling endosomes decrease in number through the time-course. Nanosensors also have been shown to accumulate in late endosome/lysosomal vesicles rather than being rapidly eliminated from the cells as seen at 37° C.

The ability to slow down endocytosis at 25° C. versus 37° C. was demonstrated. At 25° C. there was a trend of an almost linear increase in nanosensors to 1200 at about 50 minutes, followed by an almost linear decrease out to approximately 400 at 80 minutes. When this data were compared to the cells incubated at 37° C., it was seen how rapid the sensors traffic through the endocytic pathway at the higher temperature. The nanosensors demonstrated a rapid decrease from 85 nanosensors at three minutes to approximately 20 at 15 minutes, followed by a slower decrease from 20 at 15 minutes to five at 40 minutes.

The nanosensors demonstrated the temperature effects that occur during IgE receptor mediated endosomal vesicle trafficking when cells deviate away from the normal incubation temperature of 37° C. to 25° C. The nanosensor number per cell is much lower at 37° C. than at 25° C.

### Chemical Stimulation

Nanosensors were also challenged chemically by determining the effects of H⁺ flux on pH and IgE receptor mediated endosomal vesicle trafficking. The first drug used was amiloride, a known inhibitor of the Na⁺/H⁺ exchanger, blocking Na⁺ entry across the membrane and further blocking exit of H⁺ ions. Without being limited by theory, the blockage of H⁺ exit from the endosomal vesicle is believed to cause a decrease in endosomal pH, leading to vesicles with an acidic pH. Images without chemical treatment showed a relatively even dispersion of nanosensor in the various pH groupings. The untreated cells showed 50 to 55% of nanosensors in the late endosomal and lysosomal pH range at sixty and a half minutes. The amiloride-treated cells showed approximately 90% of nanosensors in the pH 4.5-6.0 range at thirty nine and a half minutes and all of the nanosensors at sixty and a half minutes in the pH 4.5-6.0 grouping or late endosomal/lysosomal stage. The images with amiloride addition demonstrated a rapid decrease of pH in endosomal vesicles, far more extreme than in untreated cells.

It has been demonstrated that amiloride addition to RBL cells shows a two fold decrease in cellular pH recovery with a decrease in pH from the initial pH before drug treatment. The overall decrease in endosomal pH would be expected if Na⁺ was unable to traverse the endosomal membrane in exchange for H⁺, further trapping H⁺ within the endosome and causing a lower pH. Amiloride causes a rapid decrease in nanosensor pH and does not show whether the nanosensors are rapidly transported through endosomal vesicles typically having a low pH, or whether the nanosensors are progressing through endocytosis with decreased endosomal vesicle pH. The relative nanosensor number per cell at given time points and the number of nanosensors in untreated cells was examined. A rapid accumulation was found of 1,000 nanosensors at fifteen minutes and 1,300 at thirty minutes, in amiloride treated cells. The untreated cells reached 1,300 nanosensors at approximately forty-five minutes. During the forty-five minute images to sixty minute images, the untreated cells started to decrease in the number of nanosensors. The amiloride treated cells at forty-five minutes and sixty minutes still demonstrated 1,300 nanosensors per cell.

The data demonstrated that amiloride treatment on the cell causes a rapid internalization of nanosensors, similar to untreated, and a marked decrease in vesicle pH, and a block in nanosensor elimination from the cell. The nanosensor numbers do not represent an exact 1:1 ratio to pixel number, but at least one nanosensor is present at each pixel, with a 1:1 correlation of pixel intensity to SEAS signal. The evidence showed that it is likely that the nanosensors are rapidly endocytosed in vesicular compartments with a low pH. Endosomal vesicle progression could be affected by the large drop in pH in the early stages of endocytosis. Incubation of cells at 25° C. slowed down vesicle progression enough to enhance the effects of amiloride on vesicle pH and demonstrate the inhibited recovery of the H⁺ pumps.

Another approach to looking at the effects of amiloride on endosomal pH was to add the drug to the cell for two hours prior to addition of nanosensors and imaging. The effects of amiloride on nanosensor pH were compared to untreated cells. The data with amiloride added to the cells in comparison to the untreated cells were similar. The data represented up to 30 minute time points. Cellular treatment with amiloride at 37° C. did not have a dramatic effect on causing an increased number of nanosensors in the pH 4.5-5.5 pH groupings as seen in the cells incubated at 25° C. A similar pattern was observed in the untreated cells and the amiloride treated cells with respect to late endosomal/lysosomal pH range of 4.5-5.5. The untreated cells showed 70% of nanosensors in this category at twelve minutes and amiloride treated cells show 60% in the pH 4.5-5.5 range. At thirty minutes the untreated cells showed 30% of nanosensors in the pH 4.5-5.5 range and the amiloride treated cells showed 55% of nanosensors in this grouping. The presence of amiloride showed an effect causative to the decreased endosomal vesicle pH persistence over time.

At 37° C. the rapid decrease in nanosensor pH causative of amiloride is not demonstrated as was in the 25° C. incubated cells. After two hours of incubation with amiloride, the cells show a mild effect on endosomal vesicle pH with an increase of nanosensors in the pH 4.5-5.5 range. It was also shown that as the untreated cells demonstrated a decrease in the percentage of nanosensors displaying pH from 6.0-7.0, the amiloride treated cells still displayed a persistence of nanosensors with more neutral pH.

To study whether amiloride is effective over time, amiloride was added and then removed from the cells and replaced with fresh Hanks solution without amiloride. The cells were incubated at 37° C. with nanosensors for two hours without amiloride. Amiloride was added to the cells. The addition of amiloride demonstrated a rapid increase in nanosensors in the pH 4.5-5.5 range to 60% from 45% without amiloride addition. Twelve minutes after amiloride addition, the effects of the drug seem to be eliminated as the pH groupings recovered to look more like the untreated cells. It is stated in the literature that amiloride addition to cells results in a two fold decrease in cellular pH recovery. The data indicated that amiloride addition to the cells inhibits the Na⁺/H⁺ exchanger, causing a decreased pH in endosomal vesicles.

Another drug that was used to assess the effects on pH due to H⁺ flux inhibition was bafilomycin. Similar to amiloride, bafilomycin was added to the cells at the same time as the nanosensors, and the cells were incubated at 25° C. Over the sixty and a half minute time course, the percentage of nanosensors in the pH grouping of 4.5-5.5 increased to 40% from 30% at thirty nine and a half minutes, when treated with bafilomycin. As a comparison, the untreated cells displayed 30% of nanosensors in the pH grouping of 4.5-5.5 at thirty nine and a half minutes followed by an increase to 32% at sixty minutes. The dramatic effect of bafilomycin on endosomal pH is demonstrated in the pH grouping from 5.5-6.0, where nanosensors could be accumulating in early to late endosomal vesicles. The effect of bafilomycin on blocking the delivery of molecules to late endosomes and lysosomes has previously been shown in cases where endosomal acidification is affected. At sixty and a half minutes the bafilomycin-treated cells had 45% of their nanosensors in the 5.5-6.0 pH grouping, as opposed to the untreated cells at sixty and a half minutes that had only 25% of their nanosensors in this pH grouping. The data demonstrated that bafilomycin addition to cells at 25° C. is effective in causing an increase in nanosensors in the pH range of 5.5-6.0, and not having an effect on early endosomal vesicles.

The total relative nanosensor number was plotted at each time point in the untreated cells and in the bafilomycin-treated cells. The data demonstrated that bafilomycin-treated cells displayed a rapid increase in the number of nanosensors, reaching a maximum at fifteen minutes. The untreated cells demonstrated the maximum amount of nanosensors after forty-five minutes. In the bafilomycin-treated cells at thirty minutes the number of nanosensors started to decrease and is at 80% of maximum at sixty minutes. The number of nanosensors in bafilomycin-treated cells was lower at a maximum of 250 nanosensors per cell and the untreated cells reached a maximum of 1,200 nanosensors per cell.

Looking at cellular effects due to bafilomycin at 25° C., the effects at 37° C. also were studied. For these experiments the drug was added to the cells for two hours prior to addition of nanosensors. With the cells incubated at 37° C., the effects of bafilomycin on endosomal pH were compared to untreated cells. The untreated cells demonstrated a rapid decrease of nanosensors in pH grouping 4.5-5.5 from twelve minutes to thirty minutes. The bafilomycin-treated cells demonstrated an accumulation of nanosensors in the pH grouping 4.5-5.5, starting with 20% of nanosensors in the same grouping at twelve minutes to 35% of nanosensors in that grouping at thirty minutes. The bafilomycin-treated cells had 55% of their nanosensors in the 6.0-7.5 pH grouping at twelve minutes and at thirty minutes there were still 35% of nanosensors within that pH grouping.

Cellular inhibition of endosomal acidification has been shown to decrease intracellular recycling and increase retention of receptors. The untreated cells have a significantly lower amount of nanosensors in the pH 6.0-7.5 early sorting and recycling endosomal pH grouping of 10% at twelve minutes. At thirty minutes, the untreated cells show only 20% of the nanosensors in the pH 6.0-7.5 grouping. Bafilomycin is expected to block the H⁺ ATPase pump and cause an increase in endosomal vesicle pH.

The total number of nanosensors at a given time point was determined in order to demonstrate the effect of bafilomycin on vesicle progression. At twenty five minutes, indicated bafilomycin was removed from the media and replaced with Hanks solution. The number of nanosensors was steady over the twenty five minutes with bafilomycin ranging from 120 to 180 nanosensors. Removal of bafilomycin from the media caused a 65% decrease in nanosensors in the first five minutes after removal. The rapid decrease in nanosensors leads to the conclusion that bafilomycin acts to raise the vesicular pH and functions in blocking nanosensors along the endocytic pathway. Once bafilomycin was removed, the number of nanosensors rapidly decreased, indicative of elimination of nanosensors from the cell.

Cells were further incubated at 37° C. with nanosensors for two hours without the addition of bafilomycin and then with bafilomycin. The addition of bafilomycin demonstrated a progressive increase of nanosensors in endosomes with pH 4.5-5.5. Untreated cells displayed only 20% of nanosensors in endosomes with pH 4.5-5.5 vs. the increase to 35% after addition of bafilomycin to almost 55% at 30 minutes. The data indicated a block in endocytic progression with nanosensors accumulating in the late endosomal and lysosomal stage.

In all embodiments of the present invention, all ranges are inclusive and combinable. All numerical amounts are understood to be modified by the word “about” unless otherwise specifically indicated. All documents cited in the Detailed Description of the Invention are, in relevant part, incorporated herein by reference; the citation of any document is. not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of simultaneously sensing and probing a biological system comprising:
a) providing a nanoparticle composite comprising:
i. a nanoparticle comprising a core and a shell;
ii. a first ligand bound to the nanoparticle, said first ligand capable of sensing pH;
iii. a second ligand bound to the nanoparticle, said second ligand distinct from said first ligand and capable of binding to a target;
b) bringing the nanoparticle composite into contact with the biological system to produce a first and a second pH-dependent signal; and
c) analyzing the first and the second signal by means of surface-enhanced Raman spectroscopy.

2. The method of claim 1, wherein said core comprises gold.

3. The method of claim 1, wherein said shell comprises silver.

4. The method of claim 1, wherein said nanoparticle is a composite nanoparticle.

5. The method of claim 1, wherein the first ligand is 4-mercaptopyridine.

6. The method of claim 1, wherein the second ligand is a 1,2-ε-dinitrophenol-L-lysine conjugate.

7. The method of claim 9, wherein the pH is from about 4.0 to about 8.0.

8. The method of claim 1, wherein the target is an IgE receptor.

9. The method of claim 1, wherein the pH-dependent signal changes in response to at least one external stimulus.

10. The method of claim 9, wherein the external stimulus is temperature.

11. The method of claim 9, wherein the external stimulus is amiloride, bafilomycin, or combinations thereof.

12. A nanoparticle composite comprising:
a) a nanoparticle comprising a core and a shell;
b) a first ligand bound to the nanoparticle, said first ligand capable of sensing pH; and
c) a second ligand bound to the nanoparticle, said second ligand distinct from said first ligand and capable of binding to a biological target.

13. The nanoparticle composite of claim 12, wherein said core comprises gold.

14. The nanoparticle composite of claim 12, wherein said shell comprises silver.

15. The nanoparticle composite of claim 12, wherein said nanoparticle is a composite nanoparticle.

16. The nanoparticle composite of claim 12, wherein the first ligand is 4-mercaptopyridine.

17. The nanoparticle composite of claim 12, wherein the second ligand is a 1,2-ε-dinitrophenol-L-lysine conjugate.

18. The nanoparticle composite of claim 12, wherein the pH is from about 4.0 to about 8.0.

19. The nanoparticle composite of claim 12, wherein said nanoparticle is suitable for use in analysis by surface-enhanced Raman spectroscopy.

20. A nanoparticle composite comprising:
a) a nanoparticle comprising a noble metal;
b) a first ligand bound to the nanoparticle, wherein the first ligand is 4-mercaptopyridine;
c) a second ligand bound to the nanoparticle, wherein said second ligand is a 1,2-ε-dinitrophenol-L-lysine conjugate;
and wherein the nanoparticle composite is suitable for use in analysis by surface-enhanced Raman spectroscopy.
